(19) United States
(12) Patent Application Publication
(10) Pub. No.: US 2013/0041164 A1
(43) Pub. Date: Feb. 14, 2013

(54) Reduction of Alpha-Tocopherol Quinone

(71) Applicants: David K. Ryan, Reading, MA (US); Isso Bayala, Worcester, MA (US); Eugene Rogers, Pepperell, MA (US)

(72) Inventors: David K. Ryan, Reading, MA (US); Isso Bayala, Worcester, MA (US); Eugene Rogers, Pepperell, MA (US)

(21) Appl. No.: 13/643,812

(22) PCT Filed: Apr. 29, 2011
(86) PCT No.: PCT/US11/34584

§ 371 Date: Oct. 26, 2012

Related U.S. Application Data
(60) Provisional application No. 61/329,555, filed on Apr. 29, 2010.

(51) Int. Cl. C07D 311/74

(52) U.S. Cl. 549/408

(57) ABSTRACT

α-tocopherol quinone is chemically reduced by combination with a reducing agent, such as tin (II) ion in the form of stannous chloride (SnCl₂-2H₂0), or by chromium (III) ion, such as chromium (III) in the form of chromium chloride (CrCl₃-6H₂0). Purified α-tocopherol is obtained from α-tocopherol formed by reduction of an oxidized α-tocopherol, such as α-tocopherol quinone, by tin (II) ion or chromium (III) ion. Purified α-tocopherol of the invention can be administered to patients in need thereof, α-tocopherol is preserved by combination with a reducing agent.

### RELATED APPLICATION

This application claims the benefit of U.S. Provisional Application No. 61/329,555, filed on Apr. 29, 2010. The entire teachings of the above application are incorporated herein by reference.

### BACKGROUND OF THE INVENTION

Vitamin E represents a family of eight antioxidants. Four of them are tocopherols (TOH) (alpha, beta, gamma, delta). Another four are tocotrienols (also alpha, beta, gamma and delta). Most of the research published so far on vitamin E suggest that α-tocopherol (α-TOH) is the only one of the family to have significant nutritional value. In addition, α-TOH contains three chiral centers (the 2, 4′, and 8′ carbons), each of which is a carbon atom with four uniquely different substituents, and as such has eight possible stereoisomers. Each chiral center has two possible arrangements in space of the substituent groups and is designated either R or S. Thus α-TOH can have RRR, RRS, RSS, RSR, SRR, SRS, SSR or SSS stereoisomers that are possible, where the R and S designations refer to the 2, 4′, and 8′ position, respectively. When α-TOH is synthetically prepared, all eight forms are present in roughly equal amounts and the mixture is called “racemic”. The α-RRR TOH form is the one predominantly formed in nature and this form is also the most biologically active. A tocopherol binding protein present in humans and animals is responsible for the preferential absorption and distribution of α-tocopherol throughout the body.

During the course of normal metabolism and when the body is exposed to smoke, pollutants or radiation, free radicals are formed. Body fat is vulnerable to destruction through oxidation by free radicals. α-Tocopherol, a fat soluble vitamin, plays a special role in eliminating free radicals and prevents a chain reaction of lipid destruction.

Common sources of vitamin E are vegetable oils, nuts, green leafy vegetables and fortified cereal. Exposure to light, oxygen, heat and chemicals can lower the content of vitamin E in foodstuffs, sometimes up to 50% during storage. Commercial vitamin E is obtained by distillation, methylation, and esterification. During production and sometimes during storage, vitamin E is oxidized. One of the major oxidation products is α-tocopherol quinone (“TQ”), which has no known antioxidant properties. Commercial vitamin E is often sold in the form of all-rac-α-tocopheryl acetate and all-rac-α-tocopheryl succinate because they are more resistant to oxidation. However, all-rac-α-tocopheryl succinate has a lower absorption coefficient compared to that of the α-tocopheryl acetate. That difference can be explained by the ability of the pancreas to hydrolyze α-tocopheryl acetate more quickly than all-rac-α-tocopheryl succinate.

Therefore, a need exists to overcome or minimize the above-referenced problems.

### SUMMARY OF THE INVENTION

The invention generally is directed to a method of producing α-tocopherol, and in particular RRR-α-tocopherol, from α-tocopherol quinone that originated by oxidation of α-tocopherol, or specifically RRR-α-tocopherol.

In one embodiment, the invention is a method of producing α-tocopherol by contacting α-tocopherol quinone with a tin (II) ion or chromium (III) ion, to thereby reduce the α-tocopherol quinone to α-tocopherol. The tin (II) or chromium (III) can be present in the form of a dissolved salt. The tin can be present in the form of dissolved stannous chloride (SnCl₂.2H₂O) or the chromium is in the form of dissolved chromium chloride hexahydrate (CrCl₃.6H₂O). The α-tocopherol quinone may also be part of a mixture (i.e. not isolated or purified) and be combined with tin (II) ion or chromium (III) ion to form α-tocopherol.

In another embodiment, the invention is a method of preventing the oxidation of α-tocopherol to α-tocopherol quinone by combining α-tocopherol with a tin (II) or chromium (III) ion.

α-tocopherol quinone is formed during the oxidation of α-tocopherol (which is primarily all natural RRR-α-tocopherol) during vegetable oil processing and purification. Large quantities of the quinone become part of a waste stream and some may end up in the soap stock and are available for very low prices. Reduction back to the RRR-α-tocopherol form in good yield converts a substance of very low cost to one that is several orders of magnitude higher in value.

By reacting α-tocopherol quinone with stannous chloride, or a salt of tin (II), in an organic solvent, such as methanol, up to 95% of the α-tocopherol quinone can be reduced to form active vitamin E. Also, stannous chloride or other tin (II) salt can be employed to prevent the oxidation of α-tocopherol even when exposed to a powerful oxidation catalyst such as CuCl₂. Likewise reacting α-tocopherol quinone with chromium chloride, or a salt of chromium (III), in ethanol, up to 92% of the α-tocopherol quinone can be reduced to form active vitamin E.

### BRIEF DESCRIPTION OF THE DRAWINGS

FIG. 1 is an HPLC chromatogram of α, β, γ and δ-TOH standards (a) and α-tocopherol produced by a reduction reaction of α-TQ standard and SnCl₂.2H₂O (b).

FIG. 2 is a MALDI-TOF mass spectrum of standard α-TOH (a) and α-TOH produced by a reduction reaction of standard α-TQ and SnCl₂.2H₂O (b), wherein the parent peak of standard α-TOH has a mass-to-charge ratio (m/z) of 430.

FIG. 3 is an HPLC chromatogram of standard α-TQ (a) and TQ produced by oxidizing α-TOH in the presence of CuCl₂ (b).

FIG. 4 is an HPLC chromatogram of standard α-TOH (a) and α-TOH produced by reaction of SnCl₂.2H₂O and TQ, wherein the TQ was obtained by oxidizing a commercial standard of α-TOH in the presence of CuCl₂.

FIG. 5 is a MALDI-TOF mass spectrum of standard α-TOH (a) and α-TOH produced by reduction of TQ, wherein the TQ is produced by oxidizing a commercial standard α-TOH in the presence of CuCl₂, and wherein the m/z peak of α-TOH is 430.

FIG. 6 is an HPLC calibration curve of α-TOH peak areas plotted versus concentration.

FIG. 7 is an HPLC calibration curve of α-tocopherol quinone peak areas versus concentration.

FIG. 8 is an HPLC chromatogram of α-TOH (a), α-TOH treated with CuCl₂ (b), α-TOH treated with SnCl₂.2H₂O (c), and α-TOH treated with both CuCl₂ and SnCl₂.2H₂O (d).

FIG. 9 is an HPLC chromatogram of α-TOH produced by reactions between TQ standard and CrCl₃.6H₂O (a), CrCl₃.6H₂O and ethanol (b), and TQ standard and ethanol (c).

FIG. 10 is an HPLC chromatogram of α-, γ-, and δ-TOH standards (a) and α-TOH produced by the reduction of the TQ standard with CrCl₃.6H₂O (c).

FIG. 11 is a MALDI-TOF mass spectrum of standard α-TOH (a) and α-TOH produced by TQ reduction with CrCl₃.6H₂O (b).

FIG. 12 is an HPLC chromatogram of standard α-TQ (a), TQ from non-spiked oil waste (b), and TQ from spiked oil waste (c).

FIG. 13 illustrates a scheme for processing vegetable oil.

### DETAILED DESCRIPTION OF THE INVENTION

The foregoing will be apparent from the following more particular description of example embodiments of the invention.

α-Tocopherol is the only member of the vitamin E family of antioxidants known to have significant nutritional value as an antioxidant that eliminates free radicals in the body and prevents chain reaction of lipid destruction. However, commercial processing of vitamin E typically results in oxidation of significant portions of α-tocopherol to α-tocopherol quinone, which has no known antioxidant properties. The α-tocopherol molecule contains three chiral centers resulting in 8 possible stereoisomers. Depending on the arrangement of functional groups around the chiral carbons, the compound is designated RRR, RRS, RSS, SSS, RSR, SRR, SSR, SRS.

The α-tocopherol quinone can be isolated from particular waste streams generated during oil processing steps that contain significant concentrations of tocopherols and tocopherol quinones. FIG. 15 is an exemplary series of refining steps for the processing of oil, such as vegetable oil. The deodorizer distillate (DOD) is one waste stream that contains significant α-tocopherol quinone. It is also likely that α-TQ is present in the soap stock especially for alkali refined oils (FIG. 15). In a chemical degumming refinery, after addition of 2 to 3% water to the crude oil, the mixture is heated to remove free fatty acids, proteinaceous materials, phosphatides, and carbohydrates in the aqueous phase by mean of settling or centrifugation. Phosphoric acid is used instead of water in the physical degumming. However the physical refinery is not suitable for all types of oils. A fundamental criterion for using this method is that the crude oils should be degummed as effectively as possible. Some types of crude oils, including palm oil and rice bran oil, contain non-hydratable phospholipids and a large amount of free fatty acids that cannot be removed by chemical refining. The degumming is followed by neutralization in the chemical refinery involving the use of caustic soda to remove resistant phospholipid complex residues and a water wash to improve the removal of the soap and phosphatides. Adsorbents such as acid-activated clays are utilized during the bleaching process to absorb color producing substances like chlorophyll and carotenoids. The final refinery process is called deodorization, during which the volatile components are removed using steam injection under high vacuum and elevated temperatures. After filtration, nitrogen is used to displace oxygen and other gases to ensure stability during the storage.

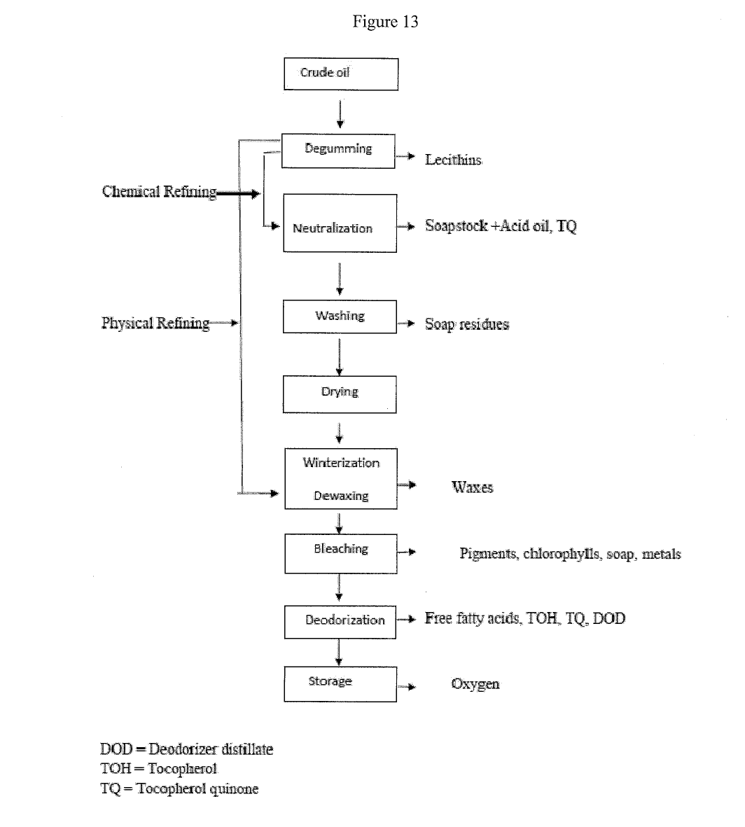

The α-tocopherol quinone can be removed from (1) the “distillate phases” (DOD, FIG. 13) collected from oil deodorization steps where compounds more volatile than the triglyceride fractions are removed and (2) “soap stocks” produced during alkali refining steps where oil is treated with base in slight excess to neutralize and removes free fatty acids and degumming agents. Included in the DOD are other ionizable and oxidized components such as tocopherol quinonone. The α-tocopherol quinone is isolated from a suitable source, such as vegetable oil, particularly vegetable oil that has been processed at elevated temperatures. Substantial amounts of α-tocopherol quinone are found in the DOD and to a lesser extent in the soap stock.

Examples of suitable methods of isolating α-tocopherol quinone or RRR-α-tocopherol quinone include liquid-liquid extraction or fractionation, gas chromatography, liquid chromatography, high performance liquid chromatography or some form of chiral chromatography, distillation or vacuum distillation. In some cases, α-tocopherol quinone may be part of an impure mixture of components (i.e. not isolated or purified) and reduced to α-tocopherol. In one embodiment, the α-tocopherol quinone isolated is a mixture of stereoisomers. In another embodiment, the α-tocopherol quinone that is isolated is essentially optically pure (i.e., contains essentially one stereoisomer of a given compound). Preferably the α-tocopherol quinone component consists essentially of RRR-α-tocopherol quinone.

Examples of suitable reducing agents include tin (II) ion and chromium (III) ion. Typically the tin (II) ion is present in the form of stannous chloride (SnCl₂.2H₂O), but essentially any other tin (II) salt will provide the reduced form of tin that will induce the reduction reaction. Likewise chromium (III) typically is in the form of chromium chloride hexahydrate (CrCl₃.6H₂O), or any salt of chromium (III).

In one embodiment, the tin (II), such as is embodied in stannous chloride, or the chromium (III), such as is embodied in chromium chloride, can be dissolved in, suspended in, or placed in contact with an organic solvent. Examples of suitable organic solvents include at least one member selected from the group consisting of methanol, ethanol, acetonitrile, hexane, chloroform, acetone, dichloromethane, and isopropanol. Preferably, stannous chloride is dissolved in methanol and chromium chloride is dissolved in ethanol. In one embodiment, stannous chloride is dissolved in methanol in a concentration range of between about 1 mg/mL and about 100 mg/mL. The dissolved stannous chloride is combined with the isolated α-tocopherol quinone in a molar ratio of stannous chloride or chromium hexahydrate: α-tocopherol quinone between about 5:1 and about 1:1. The stannous chloride solution or suspension and α-tocopherol quinone are combined by any suitable method. Typically, the temperature of the stannous chloride solution and α-tocopherol quinone is maintained in a range of between about ambient or room temperature and about 36° C. for a period of time in a range of between about 2 hours and about 12 hours during reduction of the α-tocopherol quinone by the stannous chloride.

In one embodiment, chromium chloride is dissolved in ethanol in a concentration range of between about 1 mg/mL and about 100 mg/mL. The dissolved chromium chloride is combined with the isolated α-tocopherol quinone in a molar ratio of chromium chloride: α-tocopherol quinone in a range of between about 5:1 and about 1:1. The chromium chloride solution or suspension and α-tocopherol quinone are combined by any suitable method. Typically, the temperature of the chromium chloride solution and α-tocopherol quinone is maintained in a range of between about ambient or room temperature and about 36° C. for a period of time in a range of between about 2 hours and about 12 hours during reduction of the α-tocopherol quinone by the chromium chloride.

In one embodiment, the method of the invention further includes the step of purifying the resulting reduced α-tocopherol. Examples of suitable methods of purifying the reduced α-tocopherol include at least one method selected from the group consisting of liquid-liquid extraction or fractionation, gas chromatography, liquid chromatography, high performance liquid chromatography or some form of chiral chromatography, distillation or vacuum distillation.

In another embodiment, the invention is a purified α-tocopherol composition obtained by combining α-tocopherol quinone with a suitable reducing agent, as discussed above, such as tin (II) ion (e.g., stannous chloride) or chromium (III) ion (e.g., chromium chloride). The α-tocopherol can be present, for example, in an amount in a range of between about 1% and about 100% by mass of the composition. Further, the α-tocopherol can be present in a mass ratio of α-tocopherol: α-tocopherol quinone in a range of between about 1,000:1 and about 1,000,000:1, for example. In one embodiment, the α-tocopherol present consists essentially of RRR-α-tocopherol.

In still another embodiment, the invention is a method for preserving α-tocopherol. The method includes combining α-tocopherol, such as RRR α-tocopherol, with a reducing agent, as described above.

### EXEMPLIFICATION

The invention will now be exemplified. The scope of the example is not intended necessarily to limit the invention.

### I. Materials and Methods

α-Tocopherol quinone (TQ) and α-Tocopherol (TOH) were purchased from USB Corporation (Cleveland, Ohio, USA). β-tocopherol, γ-tocopherol and δ-tocopherol were purchased from Sigma-Aldrich (St. Louis, Mo., USA). Stannous chloride dehydrate and dihydroxybenzoic acid (DHB) were obtained from Alpha Aesar (Ward Hill, Mass., USA). Cupric chloride (CuCl₂), n-hexanes, methanol, acetonitrile (both HPLC and spectrophotometric grade), ethanol (all High Performance Liquid Chromatography or HPLC and spectrophotometric grade), and glacial acetic acid were purchased from Fisher Scientific (Fairlawn, N.J., USA). Chromium (III) chloride hexahydrate (CrCl₃.6H₂O) was purchased from EM Science (Merck KGA Darmstadt, Germany). Agilent eclipse 4.6×150 mm, 5 μm C18 columns (New Castle Del., USA) with a guard column Eclipse XDB-C18, and 0.2 μm pore size syringe filters were obtained from Scientific Resources Inc. (Eatontown, N.J., USA). Cation and anion Solid Phase Extraction (SPE) tubes where purchased from Supelco (Bellefonte, Pa., USA).

### Reaction Conditions

The α-tocopherolquinone reduction process was conducted in ethanol solvent at 35° C. in a closed light proof vial maintained on a shaker at a speed of 5 rpm in order to keep the sample homogenized.

### Reverse-Phase High Performance Liquid Chromatography

Liquid chromatography was performed on a Hewlett-Packard 1090 series II Chromatograph (Brielle, N.J. USA) equipped with a manual injector Rheodyne 7725 i and a Diode Array Detector (DAD). The mobile phase was isocratic methanol:acetonitrile 90:10 (v/v) with a flow rate of 1 mL/min The column oven temperature was kept at 37° C. for all the analyses. The results were integrated and recorded using the software ChemStation II®.

### Sample Preparation

A 10 mL solution of methanol containing 0.1 g of CuCl₂ and 0.1 g of pure α-tocopherol was prepared and kept at room temperature on a shaker for 24 hours. The sample was then filtered with a 0.2 μm pore size nylon filter. Both anion and cation exchange Solid Phase Extraction (SPE) tubes were used to remove the copper ions from the sample before injection.

A pure commercial standard α-tocopherolquinone was dissolved in a 5 mL solution of ethanol containing 0.1 g of chromium (III) chloride hexahydrate and kept at room temperature on a shaker After 12 hours, the solvent was blown down to dryness with nitrogen gas. The sample was than reconstituted in 5 mL hexane and vortexed to dissolve the TOH in the hexane. Both anion and cation exchange Solid Phase Extraction (SPE) tubes were used to remove the chromium and chloride ions followed by sample filtration through a 0.2 μm filter to remove remaining impurities. The hexane solution was also blown to dryness. The sample was dissolved in 300 μL of methanol for HPLC injection. A standard solution prepared under exactly the same conditions but without CrCl₃.6H₂O was used for comparison.

### Fraction Collection

Prior to analysis, the HPLC column was equilibrated. Samples of α-TOH and α-TQ were dissolved in methanol, and the dissolved samples were run through the HPLC column one after another and also together to determine the elution time of each compound. After injection, fractions of α-TOH and α-TQ were collected in separate 100 mL Erlenmeyer flasks at their respective elution. An aliquot of each fraction was run to ensure the purity of the compound collected.

To the TQ fraction was added 0.1 g of SnCl₂.2H₂O, which was placed on a shaker set at a speed of 5 rpm for 12 hours. The solvent was blown down to dryness with nitrogen gas. The sample was reconstituted in 5 mL hexane, vortexed and filtered to remove the SnCl₂.2H₂O. The hexane solution was also blown to dryness. The sample was dissolved in 300 μL of methanol for HPLC injection. A standard solution prepared under exactly in the same conditions but without SnCl₂.2H₂O was used for comparison.

### MALDI-TOF

Measurements were conducted on a matrix assisted laser desorption and ionization (MALDI) time of flight (TOF) mass spectrometer model MALDI-LR (Waters, Milford, Mass., USA) that incorporated an automated sample plate loader and was equipped with a 337 nm nitrogen UV laser and a 2.3 m flight tube. The mass spectra were obtained with a reflectron positive ion mode by Masslynx 3.5 software.

### MALDI Matrix Preparation

A stock matrix solution was prepared (5 mg/mL) by dissolving 50 mg of 2,5-dihydroxybenzoic acid (DHB) in 10 mL of 0.1% Trifluoroacetic acid:acetonitrile in a 2:1 (v/v) ratio and sonicated for 5 min for the TOH analysis.

### MALDI Sample Preparation

A mixture of matrix and sample (1:1 v/v) was prepared and directly used on a 96 well target plate after drying in an air stream at room temperature.

### MALDI Calibration

A mixture of monoisotopic caffeine, monoisotopic perylene D12, and monoisotopic sulfonic acid were used for calibration to ensure highly accurate results.

### Statistical Analysis

In order to ensure proper instrument working conditions and accurate results, the following parameters were determined: linear range, instrument limit of detection, method precision, and accuracy. All the experiments were conducted in triplicate and results were averaged with standard deviations and relative standard deviations calculated to determine acceptable results.

### II. REDUCTION With SnCl₂.2H₂O

### Reduction of Commercial α-TQ

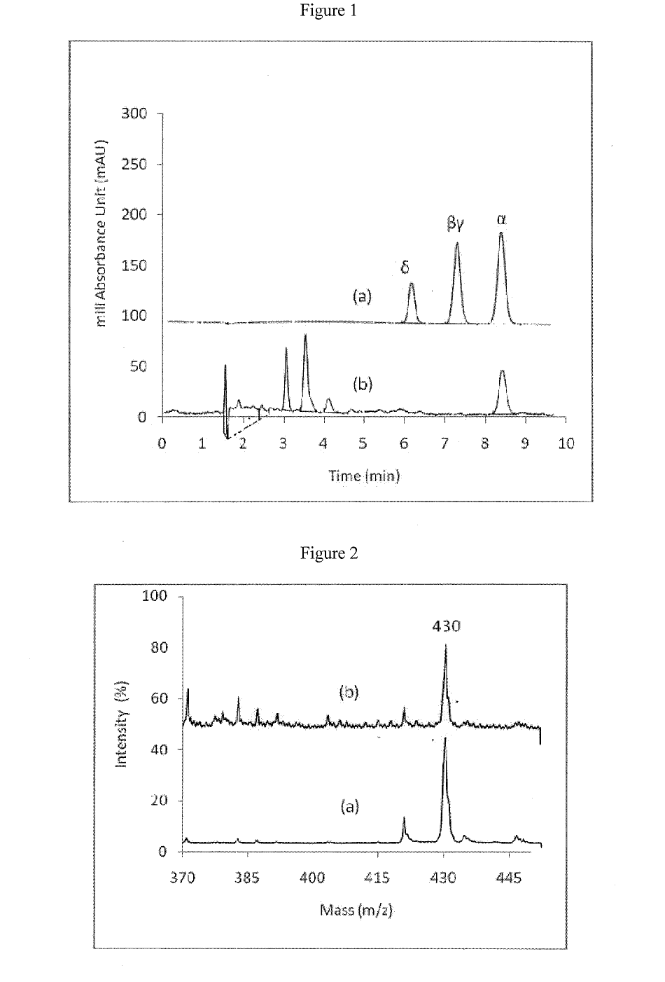

We started our experiments with an unknown concentration of commercial α-TQ and an arbitrary amount of 0.1 g of SnCl₂.2H₂O that were dissolved in a 100 mL Erlenmeyer flask containing 10 mL of methanol. The Flask was placed on a shaker set at a speed of 5 rpm in room temperature for 12 hours. The 10 mL of methanol were transferred to a 20 mL vial placed in a 35° C. water bath and blown to dryness using nitrogen gas. A 5 mL solution of hexane was then added to the vial, vortex-mixed for 10 seconds, and filtered with a 0.2 μm nylon filter to remove the SnCl₂.2H₂O. The filtrate was collected in a new 30 mL clean vial and also blown to dryness using nitrogen gas. The TQ sample was resuspended in 3000 methanol for HPLC injection. FIG. 1 is an HPLC chromatogram of α, β, γ, and δ-TOH standards (a) and α-TOH produced by reduction of α-TQ standards and SnCl₂.2H₂O. FIG. 1 shows the formation of a new compound positively identified by HPLC as α-TOH by comparing its retention time in the chromatogram (b) to the retention time of α, β, γ and δ-TOH standards in the chromatogram (a) obtained in identical conditions. The formation of three other unidentified products was also observed on the chromatogram (b) of the FIG. 1. Since the retention time of these products is between 2 and 4.5 minutes it can be deduced that they are different from both TQ and TOH.

FIG. 2 is a MALDI-TOF mass spectrum. A positive ion mode MALDI-TOF used on a commercial standard vitamin E gave a major intense peak at m/z 430 (FIG. 2, spectrum a). By matching the peak to that of the sample (FIG. 2, spectrum b), we were able to confirm the identity of the new compound as α-TOH.

### Oxidation of α-TOH by CuCl₂ in Methanol

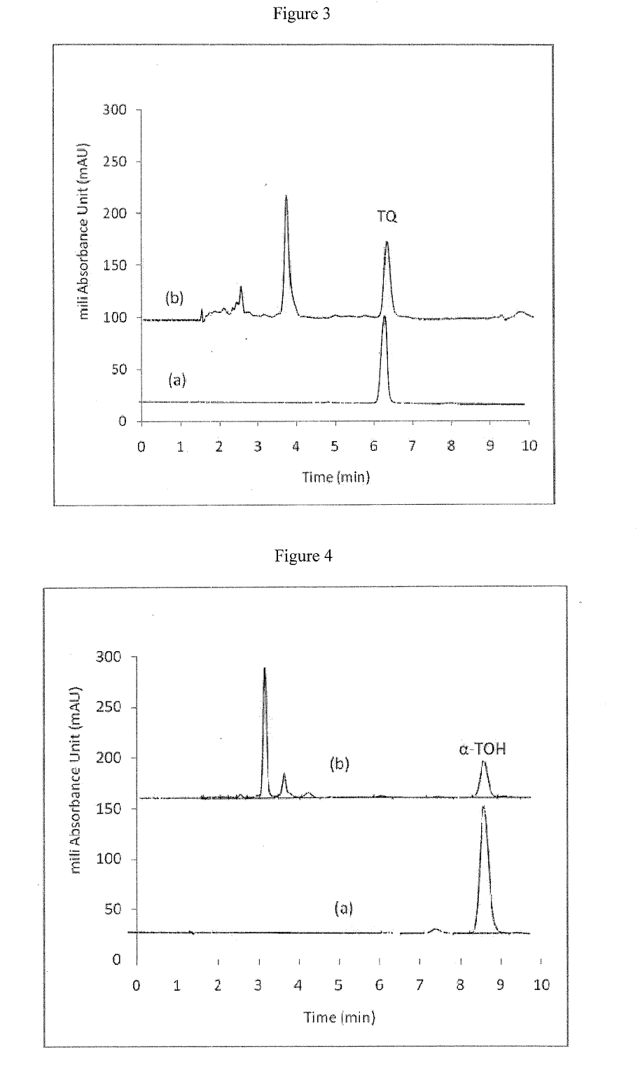

The results obtained in FIGS. 1 and 2 led us to produce TQ by oxidizing α-TOH in the presence of CuCl₂. One gram of commercial α-TOH and 1 g of CuCl₂ were dissolved in 10 mL of methanol contained in a 100 mL Erlenmeyer flask. The flask was placed on a shaker set at a speed of 5 rpm for 12 hours at room temperature. The 10 mL of methanol were transferred to a 20 mL vial after filtration through a 0.2 μm nylon filter and cleanup with an SPE tube to remove the CuCl₂. The filtrate was used for HPLC injection. FIG. 3 is an HPLC chromatogram showing the retention time of α, β, γ and δ-TOH standards (a) and TQ produced by oxidation of α-TOH in the presence of CuCl₂ (b). Chromatogram (b) shows the formation of oxidation products including a major compound identified by HPLC as TQ by comparing its retention time to that of the commercial α-TQ obtained in the same conditions (chromatogram a). The TQ was isolated in a 20 mL vial by fraction collection.

### Reduction of α-TOH Oxidation Product

The TQ fraction collection vial was blown to dryness using nitrogen gas to remove the acetonitrile of the mobile phase. The TQ sample was resuspended in 10 mL methanol solution containing 0.1 g of SnCl₂.2H₂O and placed on a shaker set at 5 rpm in room temperature for 12 hours. FIG. 4 is an HPLC chromatogram of a standard containing commercial α-TOH (chromatogram a) and the TQ sample after 12 hours on the shaker (chromatogram b). The interactions between the molecules of SnCl₂.2H₂O and TQ resulted in the formation of TOH, identified by HPLC as α-TOH.

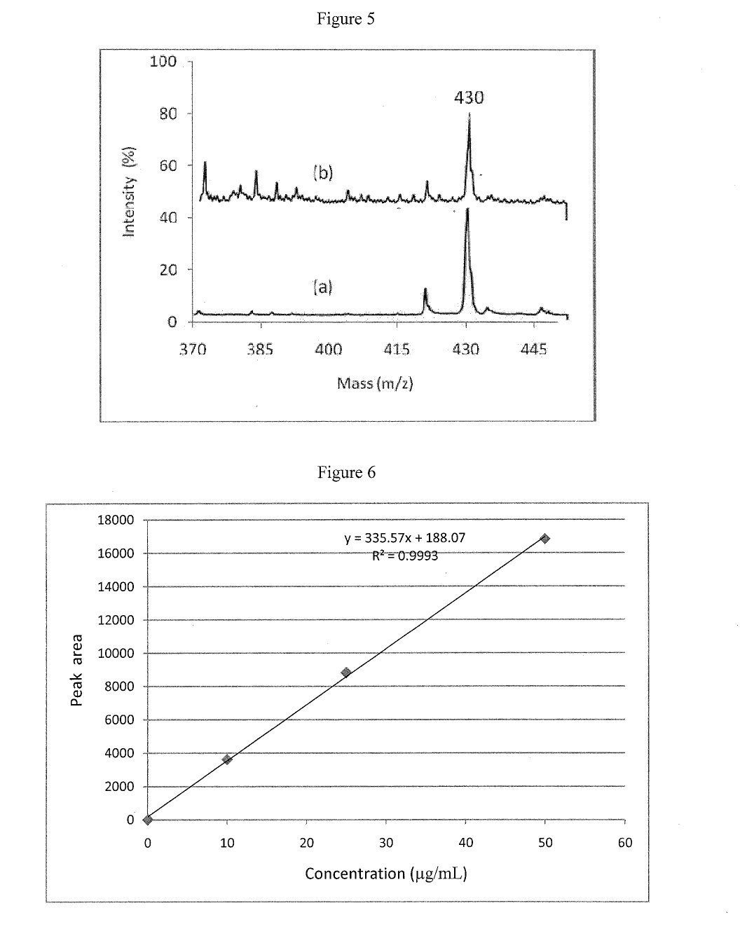

FIG. 5 is a MALDI-TOF mass spectrum of standard α-TOH (a) and α-TOH produced by the reduction of TQ previously collected (b). A positive ion mode MALDI-TOF gave a perfect match between the spectrum of the commercial standard α-TOH and the sample, as shown by the m/z peak at 430, confirming the identity of the sample α-TOH.

In order to estimate the amount of α-TOH contained in the sample injected, a linear standard calibration curve was generated using a stock solution of α-TOH prepared and diluted at the following concentrations: 0.01, 10, 25, 50 μg/mL. Each concentration was run three times to ensure a statistically-acceptable value, and the results are shown in Table 1. The high value of the correlation coefficient (R²=0.999) indicates a strong relationship between concentration and HPLC signal response for any quantity of vitamin E between 0.01-50 μg/mL (FIG. 6). The limit of detection (LOD) was experimentally determined to be 0.01 μg/mL The instrument detection limit (IDL) was calculated as the concentration of vitamin E corresponding to the smallest signal that can be distinguished from the background noise response and estimated to 0.1 ng/mL

TABLE 1
Analysis of different concentrations of α-TOH
by HPLC and their corresponding peak areas

| TOH (μg/mL) | 0.01 | 10 | 25 | 50 |
|---|---|---|---|---|
| Peak area (mean ± | 10.98 ± 0.7 | 3617.50 ± 42.5 | 8820.21 ± 117.5 | 16830.50 ± 133.3 |
| SD, n = 3) | | | | |

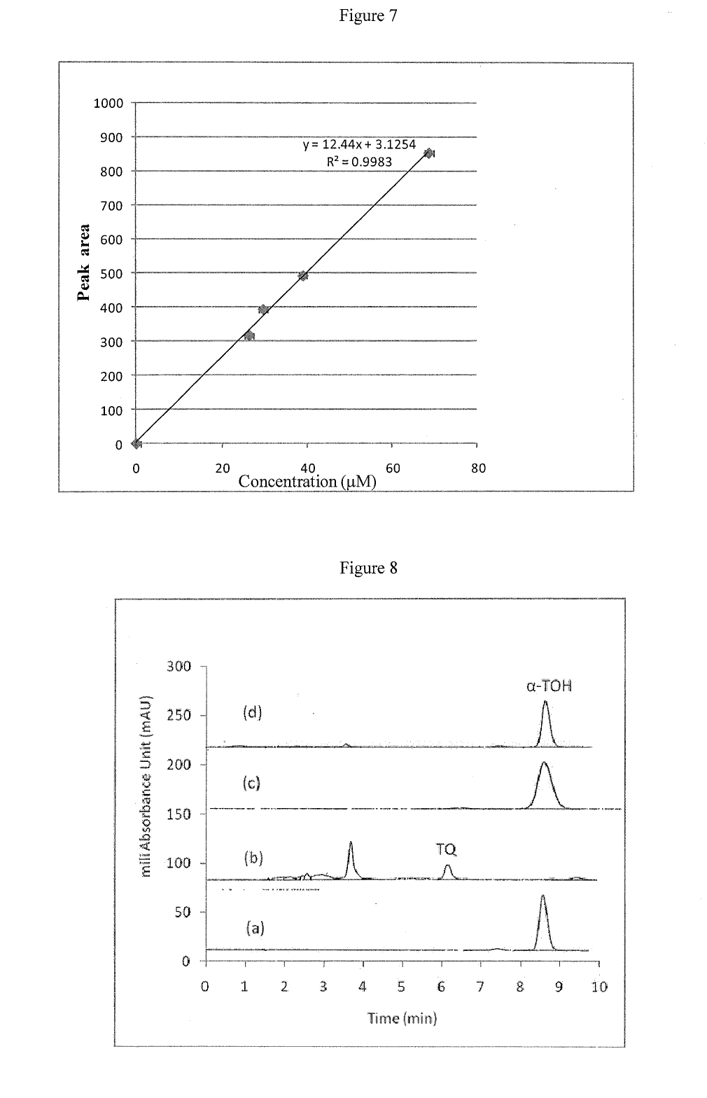

Due to the limited amount of commercial standard α-TQ stock and its high cost on the market, the molar absorptivity (TQ at λmax=268 nm, ε=18.2 mM⁻¹ cm⁻¹) was used to calculate the concentrations according to the Beer Lambert Law. FIG. 7 is a linear calibration curve obtained by plotting a graph of peak area versus concentration for a 20 μL injection of the four concentrations of α-TQ standard aliquots. The data obtained from HPLC peak integration software and TQ concentrations by UV-VIS spectrophotometer are given in the Table 2.

TABLE 2
Analysis of different concentrations of α-TQ
by HPLC and their corresponding peak areas

| TQ (μM) | 26.374 | 29.67 | 39.011 | 68.681 |
|---|---|---|---|---|
| Peak area | 315.93 ± 1.1 | 391.99 ± 1.5 | 491.86 ± 9.9 | 853.14 ± 10.8 |
| (mean ± | | | | |
| SD, n = 3) | | | | |

### Optimization

To maximize the production of α-TOH, we conducted the experiments at different temperatures and PH. The highest reduction of TQ to α-TOH was achieved when the reaction was conducted at 35° C. and the pH adjusted with concentrated glacial acetic acid to pH 4. Using the linear calibration curves of standard α-TQ (FIG. 7) and standard α-TOH (FIG. 6) the percentage of TQ reduced and quantity of α-TOH produced, as shown in Table 3.

TABLE 3
HPLC peak areas of α-TQ 95% reduced by SnCl₂•2H₂O and α-TOH
formation in methanol at 35° C. and pH 4

| Product | TQ | TOH |
|---|---|---|
| Peak area (mean ± | 832.43 ± 4.4 | 40.28 ± 0.7 |
| SD, n = 3) | | |
| Concentration | 27 mg/mL | 0.44 μg/mL |

According to the results obtained in the Table 3 a concentration of 27 mg/mL of standard α-TQ was able to generate 0.44 μg/mL of α-TOH.

### Antioxidation Effect of SnCl₂.2H₂O on TOH

In order to study the individual and collective effects of both SnCl₂.2H₂O and CuCl₂ on vitamin E, 1 g of commercial α-TOH and 1 g of CuCl₂ were dissolved in 10 mL of methanol solution contained in a 100 mL Erlenmeyer flask. The flask was placed on a shaker set at a speed of 5 rpm for 12 hours at room temperature. The 10 mL of methanol were transferred to a 20 mL vial after filtration through a 0.2 μm nylon filter and cleanup with a SPE tube to remove the CuCl₂. The filtrate was used for HPLC injection.

FIG. 8 is a chromatogram of commercial α-TOH (a) α-TOH treated with CuCl₂ (b), α-TOH treated with SnCl₂.2H₂O (c), and α-TOH treated with both CuCl₂ and SnCl₂.2H₂O(d). Chromatogram (b) in FIG. 8 shows that CuCl₂ completely oxidized TOH to TQ and other unidentified products. SnCl₂.2H₂O by itself had no effect on TOH according to the chromatogram (c). The same results are observed in the Chromatogram (d), where α-TOH was exposed to both SnCl₂.2H₂O and CuCl₂.

This experiment demonstrates the protective effect of SnCl₂.2H₂O against a powerful oxidation catalyst such as CuCl₂. In the industry, SnCl₂.2H₂O could become an efficient antioxidation agent that can be used to protect and store vitamin E.

### III. REDUCTION WITH CrCl₃.6H₂

### Reduction of Commercial α-TQ

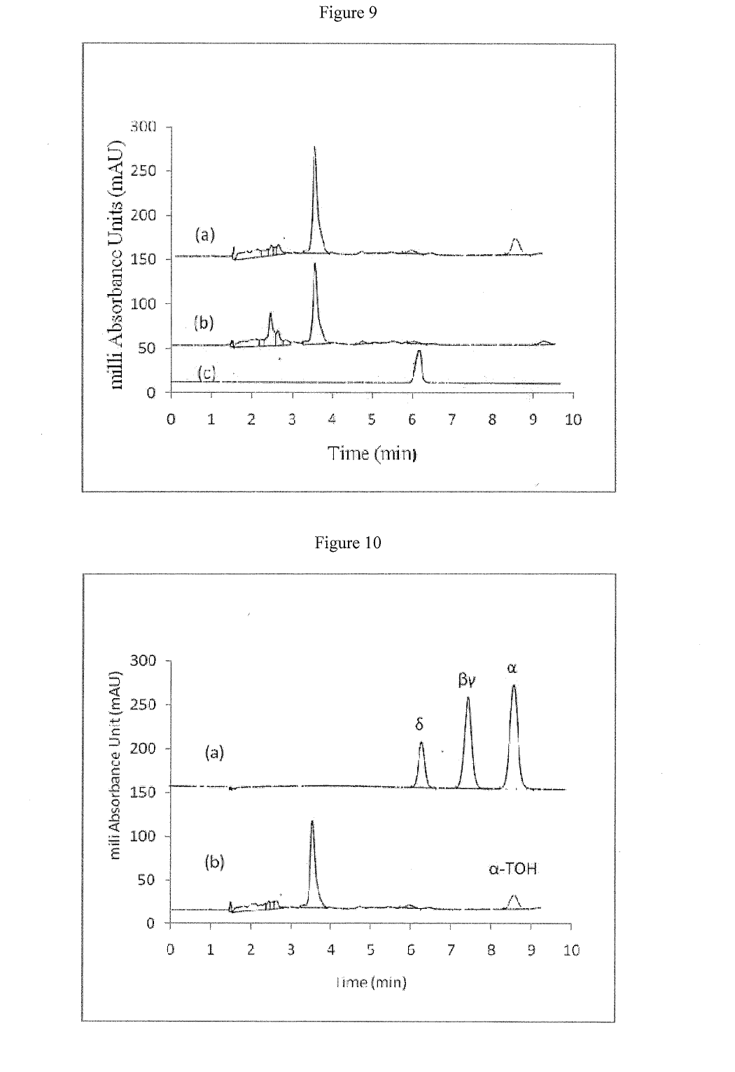

A TQ sample was prepared for HPLC injection. FIG. 9 is an HPLC chromatogram of α-TOH produced by reactions between TQ standard and CrCl₃.6H₂O (a), CrCl₃.6H₂O and ethanol (b), and TQ standards and ethanol (c). The peaks in FIG. 9 were detected at both 292 nm (maximum absorbance for TOH) and 268 nm (maximum absorbance of TQ). All three chromatograms of FIG. 9 were obtained under the exact same conditions. FIG. 9 shows the TQ is not present, in contrast to chromatogram (c), which shows the presence of TQ when CrCl₃.6H₂O is not included in the reaction mixture. Chromatogram (a) shows the production of a newly formed compound, which is not formed when CrCl₃.6H₂O (chromatogram c), positively identified by HPLC as α-TOH (see below regarding FIG. 10). The formation of other unidentified products in chromatogram (a) was probably caused by the reaction between solvent and chromium III, as determined by observing similar peaks in chromatogram (b). FIG. 10 is a chromatogram showing α, β, γ, and δ-TOH standards (a) and α-TOH produced by reactions between TQ standard and CrCl₃.6H₂O (b) (i.e., the reaction product described in chromatogram (a) of FIG. 9).

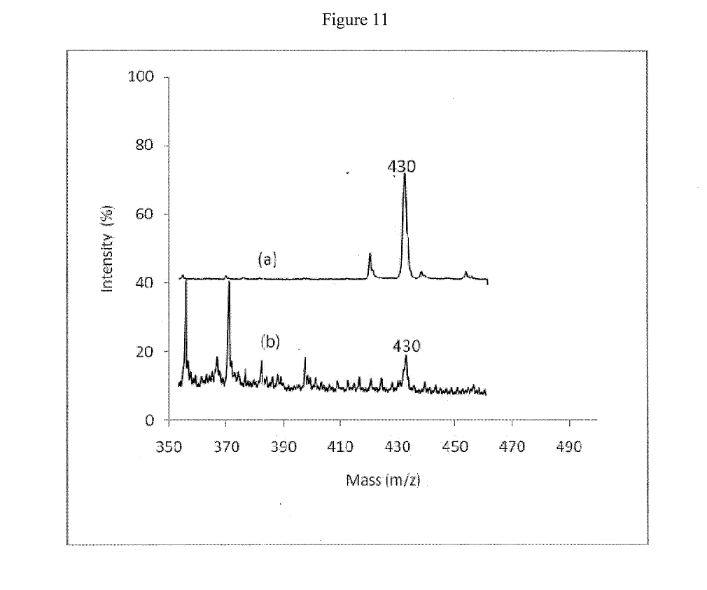

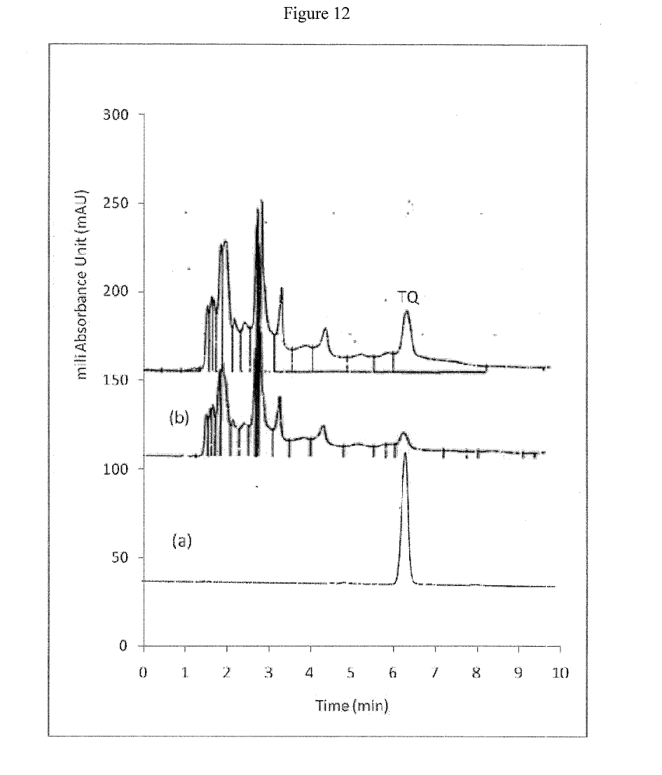

FIG. 11 is a MALDO-TOF mass spectrum of standard α-TOH (a) and α-TOH produced by TQ reduction with CrCl₃.6H₂O (b). The m/z peak of α-TOH is 430, thus confirming the identity of the compound produced as α-TOH. The presence of several other peaks may be an indication of impurities, but the difference in molecular masses helps eliminate the effect of interferences and makes it easy to demonstrate the presence of α-tocopherol as the result of reacting α-TQ with Chromium (III).

### TQ Reduction Rate

In order to estimate the percentage of TQ reduced by chromium (III) a linear calibration curve was obtained by plotting a graph of concentration versus peak area using four different concentrations of TQ standard prepared from the same stock solution (FIG. 7). The molar absorptivity (TQ at λmax=268 nm, ε=18.2 mM-1 cm-1) was used to calculate the concentrations according to the Beer-Lambert law.

In the same manner as for TQ, a linear standard calibration curve was generated using a stock solution of α-TOH prepared and diluted at the following concentrations: 0.01, 10, 25, and 50 μg/mL. The high value of the correlation coefficient (R2=0.999) indicates a strong relationship between concentration and HPLC signal response for any quantity of vitamin E between 0.01-50 μg/mL The limit of detection (LOD) was experimentally determined to be 0.01 μg/mL The instrument detection limit (IDL) was calculated as the concentration of vitamin E corresponding to the smallest signal that can be distinguished from the background noise response and estimated to be 0.1 ng/mL.

### Optimization

To maximize the production of α-TOH, we conducted the experiments at different temperatures and pH. The highest reduction of TQ was achieved when the reaction was conducted at 35° C. and the pH adjusted with glacial acetic acid to pH 4. Using the linear calibration curve of standard TQ (FIG. 7) and standard α-TOH (FIG. 6) we were able to determine the quantity of TQ reduced and the amount of α-TOH produced (Table 4).

TABLE 4
HPLC peak areas of TQ 92% reduced by CrCl₃•6H₂O and α-TOH
formation in ethanol at 35° C. and pH 4

| | TQ | TOH |
|---|---|---|
| Peak area (mean ± | 733.40 ± 4.7 | 71.86 ± 1.4 |
| SD, n = 3) | | |
| Concentration | 23.7 mg/mL | 0.35 μg/mL |

Table 4 summarizes the results of the experiments conducted in the present study: 23.7 mg/mL of TQ react with 0.1 g of CrCl_{30.6}H₂O in ethanol at 35° C. and pH 4 to produce 0.35 μg/mL of TOH.

### IV. EXTRACTION Of α-TO FROM VEGETABLE OIL DEODORIZER DISTILLATE WASTE

### Materials and Methods

The raw material was obtained as deodorizer distillate (thick viscous oily sample) from an oil refinery plant AVLON (Melrose Park, Ill., USA). All other materials are as described in Part I.

### Standard Preparation

A concentrated stock solution of pure standard TQ was prepared by dissolving TQ in 2 mL of methanol and stored in the dark at 4° C. The solution was tested prior use to ensure its stability. Working solutions were prepared from the stock by dilution in methanol immediately before HPLC injection.

### Sample Extraction and Preparation

Two series of vegetable oil waste samples of 0.5, 1.0, 1.5, 2.0, 2.5 and 3.0 g were weighed out in 12 different clear vials. The first series of 6 vials was spiked with a known amount of TQ. To dissolve the sample, 1 mL of hexane was added to each sample vial, and vortexed for 10 sec. For TQ extraction, 2 mL of acetonitrile were added to each sample vial, vortexed for 30 seconds followed by a 5 minute waiting period for raffinate and extract separation. A control made of solvents only was prepared identically to the samples in triplicate without matrix addition. The extract, characterized as the top clear solvent layer in each vial was carefully transferred to new clear vials using Pasteur pipettes. The operation was repeated two more times by adding only 2 mL of acetonitrile a second and third time to the sample vials. The transferred extracts were blown down to dryness with nitrogen gas. The content of dried vials was re-suspended in 300 μL methanol, vortex mixed, and filtered with a 0.2-μm pore size nylon filter prior HPLC injection. The amounts of TQ recovered from the control as well as from the spiked and non spiked samples were used to calculate the percentage of extractable TQ.

### Statistical Analysis

In order to insure proper instrument working conditions and accurate results, the following parameters were determined: linear range, instrument limit of detection, method precision, accuracy and peak purity. All the experiments were conducted in triplicate and results averaged with standard deviations and relative standard deviations calculated to determine acceptable results.

### Choice of Extraction Solvents

Organic solvents are commonly used in most organic compounds extractions because of their availability, convenience, and cost. Compound solubility in the solvent and separation into distinct layers constitute an important criteria in liquid-liquid extraction. In the present study, 1 mL of 7 different solvents was added to 7 transparent glass vials of 25 mL capacity containing each 1 g of oil waste in order to determine the best extraction result. Each vial was vortexed for 30 seconds followed by a 5 minute waiting period. Since the oil waste sample was thick semi-liquid, our first goal was to find a solvent in which it dissolved completely and the second goal was to select another solvent that, when mixed with the first resulted in separation into layers with the tocopherolquinone migrating to one of the solvents and the raffinate to the other solvent. Table 5 shows the results of a visual observation of the sample-solvent mixture. A one layer homogenous solution is an indication of the ability of the solvent to dissolve the oil waste completely, as oppose to the two layer heterogeneous solution, which indicates a partial dissolution.

TABLE 5
Effect of different organic solvents on oil waster

| Solvents | Matrix (g) | Results |
|---|---|---|
| Acetonitrile | 1 | + |
| Acetone | 1 | − |
| Hexane | 1 | − |
| Chloroform | 1 | − |
| Methylene chloride | 1 | − |
| Isopropanol | 1 | − |
| Methanol | 1 | + |

+ Separation (2 layers)
− no separation (homogenous)

Table 5 shows that only methanol and acetonitrile partially dissolve the oil waste. The other five organic solvents (acetone, hexane, chloroform, methylene chloride and isopropanol) have the ability of total dissolution.

Due to the complexity of the oil waste composition, its direct injection into a HPLC will plug system lines or cause particle accumulation in the column leading to interference and reduction of reproducibility. Therefore, cleaning was necessary using the liquid-liquid extraction method prior to analysis. Hexane was the first solvent of choice because of its ability to totally dissolve the oil waste. Acetonitrile and methanol were the second and third solvents of choice not only because of the ability of each of them to completely dissolve TQ but also their miscibility among themselves and immiscibility property with hexane. Using only hexane-acetonitrile or hexane-methanol did not yield high recovery (data not shown).

Vigorous vortex-mixing of oil waste-hexane-acetonitrile-methanol generated a top clear layer and a dark bottom layer after a 5 minute waiting period as a result of matrix-solvent partitioning. Since our objective is to work with cleaner samples in order to get better results and minimize instrument damage, the top clear layer was used to investigate the recovery of TQ.

### TQ Extraction

The top clear layer as described above was transferred to a new clean vial and replaced by a new 1:1 solution of acetonitrile:methanol The operation was repeated three times to ensure a maximum TQ extraction and the content of the new vial blown to dryness with nitrogen gas. The content of the dried vial was re-suspended in 300 μL of methanol for HPLC injection. The results are shown in FIG. 14, which is an HPLC chromatogram of standard α-TQ (a), TQ from non-spiked oil waste (b), and TQ from spiked oil waste (c).

The TQ was detected and identified using both chromatographic retention time and a Diode Array Detector (DAD) spectrum comparison with pure TQ standard. The sample and standard TQ were run under the same experimental conditions. FIG. 14 shows the peaks of standard TQ and the TQ present in the oil waste.

### Linearity

A calibration curve was performed to determine the linearity of 4 different concentrations of α-tocopherolquinone standard versus peak areas with the following regression equation: y=12.32x+8.726; R²=0.996 where x is the concentration of α-TQ, y the corresponding peak area and R² the correlation coefficient.

### Precision

Method precision was determined by running on the HPLC instrument six TQ standard solutions prepared individually at the same concentration level. The precision was expressed by repeatability of peak area and retention time calculated using the relative standard deviation (RSD). Results are provided in Table 6.

TABLE 6
HPLC method precision determination

| | Mean of Peak | Area RSD | Mean of Retention | Time RSD |
|---|---|---|---|---|
| n = 6 | Area | (%) | Time (min) | (%) |
| α-TQ | 4751.33 | 3.13 | 6.47 | 1.08 |

### Limit of Detection (LOD) and Limit of Quantification (LOQ)

LOD was determined using a series of different concentrations of TQ in decreasing order. The smallest HPLC peak obtained with an area five times greater than the signal to noise ratio was defined as the instrument detection limit The LOQ defined as the lowest concentration of measurable value of TQ standard solution corresponding to a peak area ten times greater than the signal to noise ratio. Both LOD and LOQ are reported in Table 7.

TABLE 7
LOD and LOQ determination

| n = 6 | LOD (ng/mL) | LOQ (ng/mL) |
|---|---|---|
| α-TQ | 0.05 | 0.1 |

### Recovery

The recovery of α-tocopherolquinone in the oil waste sample was assessed by comparing the quantity of TQ recovered from the control (solvent only spiked with TQ) to the difference between quantities of TQ extracted from both spiked and non-spiked samples (n=6). The non-spiked sample is used to extract the native TQ and the spiked one, the native plus added TQ. Table 8 shows the percentage of recovery obtained on varied amounts of matrix. A wide recovery ranging from 120% to 31.49% justifies the choice of acetonitrile, methanol and hexane as suitable solvents of choice for the extraction of TQ in oil waste. It can be deduced from the data that there is a direct correlation between higher recovery and sample-solvent ratio.

TABLE 8
TQ quantified in deodorized distillate oil waste

| Oil waste (g) | TQ recovery (%) |
|---|---|
| 0.5 | 120 ± 6.9 |
| 1 | 114.5 ± 7.64 |
| 1.5 | 106.3 ± 7.5 |
| 2 | 82.73 ± 6.88 |
| 2.5 | 41.48 ± 12.87 |
| 3 | 31.49 ± 5.91 |

The percentage of TQ recovered is proportional to the amount of solvent used for the extraction. The highest recovery was observed from 0.5 g to 2.0 g of oil waste corresponding to a ratio of matrix:solvent of 1:6 (w:v) and 1:1.5 (w:v).

### V. CONCLUSION

By conducting the present study, we were able to demonstrate that α-tocopherol quinone (TQ) can react in methanol in the presence of tin (II) to produce vitamin E under laboratory conditions at room temperature. If applied in a commercial setting where α-TOH is being isolated, these results can boost the production of vitamin E and significantly reduce the volume of vegetable oil wastes caused by the oxidation during the manufacturing process. This study has also has proved the efficiency of tin (II) in protecting vitamin E against oxidation.

### EQUIVALENTS

While this invention has been particularly shown and described with reference to example embodiments thereof, it will be understood by those skilled in the art that various changes in form and detail may be made therein without departing from the scope of the invention encompassed by the appended claims. For example, the invention has been described in detail with reference to reduction of α-tocopherol quinone to α-tocopherol, and the oxidation of α-tocopherol to α-tocopherol quinone. However, the examples are equally applicable to any member of the tocopherol family, such β-tocopherol, γ-tocopherol, and δ-tocopherol.

### INCORPORATION BY REFERENCE

The relevant teachings of all patents, published applications and references cited herein are incorporated by reference in their entirety.

What is claimed is:

1. A method of reducing the oxidation state of α-tocopherol quinone comprising the step of contacting the α-tocopherol quinone with a tin (II) ion or chromium (III) ion.

2. The method of claim 1, wherein the tin (II) or chromium (III) are present in the form of a dissolved salt.

3. The method of claim 2, wherein the tin is present in the form of dissolved stannous chloride (SnCl₂.2H₂O) or the chromium is in the form of dissolved chromium chloride hexahydrate (CrCl₃.6H₂O).

4. The method of claim 3, wherein the stannous chloride (SnCl₂.2H₂O) or the chromium chloride hexahydrate (CrCl₃.6H₂O) are dissolved in an organic solvent.

5. The method of claim 4, wherein the organic solvent is at least one member selected from the group consisting of methanol, ethanol, acetonitrile, hexane, chloroform, acetone, dichloromethane, and isopropanol.

6. The method of claim 5, wherein stannous chloride (SnCl₂.2H₂O) is dissolved in methanol.

7. The method of claim 5, wherein chromium chloride hexahydrate (CrCl₃.6H₂O) is dissolved in ethanol.

8. The method of claim 5, wherein the concentration of stannous chloride (SnCl₂.2H₂O) or chromium chloride hexahydrate (CrCl₃.6H₂O) is in a range of between about 1 mg/mL and about 100 mg/mL.

9. The method of claim 5, wherein the temperature of the stannous chloride (SnCl₂.2H₂O) or the chromium chloride hexahydrate (CrCl₃.6H₂O) and α-tocopherolquinone is maintained in a range of between about ambient or room temperature and about 36° C. for a period of time in a range of between about 2 hours and about 12 hours.

10. The method of claim 5, wherein the molar ratio of the stannous chloride (SnCl₂.2H₂O) or the chromium chloride hexahydrate (CrCl₃.6H₂O) to α-tocopherolquinone at the beginning of the reaction is in a range of between about 5:1 and about 1:1.

11. The method of claim 1, wherein the reducing agent is stannous chloride (SnCl₂.2H₂O).

12. The method of claim 11, wherein the stannous chloride is dissolved in methanol.

13. The method of claim 1, wherein the reducing agent is chromium chloride hexahydrate (CrCl₃.6H₂O).

14. The method of claim 13, wherein the chromium chloride hexahydrate (CrCl₃.6H₂O) is dissolved in ethanol.

15. The method of claim 1, wherein the α-tocopherolquinone is a component of a composition that is a racemic mixture of tocopherols.

16. The method of claim 15, further including the step of purifying the reduced α-tocopherols.

17. The method of claim 16, wherein the reduced α-tocopherols are purified by at least one method selected from the group consisting of liquid-liquid extraction, chromatographic separation, vacuum distillation.

18. The method of claim 1, wherein the α-tocopherol quinone is RRR α-tocopherol quinone.

19. The method of claim 1, further comprising the step of extracting the α-tocopherol quinone from a waste stream generated during oil processing.

20. The method of claim 19, wherein the waste stream includes a distillate phase collected from oil deodorization.

21. The method of claim 19, wherein the waste stream includes a soap stock produced during alkali refining, wherein oil is treated with a base in slight excess to thereby neutralize and remove free fatty acids and degumming agents.

22. A method of preventing the oxidation of α-tocopherol to α-tocopherol quinone, comprising the step of combining the α-tocopherol with a tin (II) or chromium (III).

23. The method of claim 22, wherein the reducing agent is stannous chloride (SnCl₂.2H₂O) or chromium chloride hexahydrate (CrCl₃.6H₂O).

24. The method of claim 23, wherein the α-tocopherol is RRR α-tocopherol.
